(19) United States
(12) Patent Application Publication
(10) Pub. No.: US 2020/0291337 A1
(43) Pub. Date: Sep. 17, 2020

(54) MICROBIOLOGICAL CULTURE PLATE

(71) Applicant: Soft Cell Biological Research, LLC, St. George, UT (US)

(72) Inventor: John Brent Hunt, St. George, UT (US)

(73) Assignee: Soft Cell Biological Research, LLC, St. George, UT (US)

(21) Appl. No.: 16/753,140

(22) PCT Filed: Oct. 5, 2018
(86) PCT No.: PCT/US2018/054762

§ 371 Date: Apr. 2, 2020

Related U.S. Application Data
(60) Provisional application No. 62/568,598, filed on Oct. 5, 2017.

(51) Int. Cl. C12M 1/22; C12M 1/00

(52) U.S. Cl. CPC C12M 23/10; C12M 29/04; C12M 23/38

(57) ABSTRACT

This disclosure relates to microbiological culture plates configured for providing hydrologic stability to an inoculant/sample placed upon solid media held within the plate. A culture plate includes a base section and a lid section shaped to fit upon and enclose the base section. The lid section includes an extension extending downward from an upper surface, and a cover attached to the lower end of the extension. The cover is configured so that when the lid section is placed upon the base section, the cover contacts the inoculant/sample placed upon the solid media in the base section.

### RELATED APPLICATIONS

This application is a national stage filing under 35 U.S.C. § 371 of international PCT application, PCT/US2018/054762, filed Oct. 5, 2018, which claims priority under 35 U.S.C. § 119(e) to U.S. provisional patent application, U.S. Ser. No. 62/568,598, filed Oct. 5, 2017, the entire contents of each of which are which is incorporated herein by reference.

### BACKGROUND

Microbial cultures may be grown in liquid/broth culture media or on solid media. When grown on solid media, microbial cultures are typically grown on culture plates containing agarose gel (“agar”) or other such gelling agent. Although liquid media can produce good growth, isolating a pure culture of a microorganism typically requires plating a sample on solid media.

Standard culture plates typically include a base with a round dish shape and an attached sidewall for providing sufficient depth to hold the agar. After contacting a sample to the upper surface of the exposed agar, a corresponding lid is usually fitted on top of the base to prevent contamination of the agar surface. A sealing tape is often wrapped around the side of the plate to seal the interface between the plate bottom and the lid.

### SUMMARY

Aspects of the disclosure relate to microbiological culture plates and methods of using the same. The disclosure is based, in part, on microbiological culture plates that are configured to (e.g., comprise or consist of one or more features that) facilitate or enhance culture of certain types of microorganisms, such as L-form bacteria (e.g., relative to standard microbiological culture plates).

Although standard culture plates are sufficient for many microbial culture applications, several limitations remain. For example, some samples may include microorganisms that exist and grow in a given cell or sample and yet are unable to be grown under the particular conditions of the solid media of the plate. Such microorganisms may therefore go undetected under standard culturing protocols. This can lead to missed diagnoses of microbial infections, underreporting of microbial populations, and mischaracterizations of microbial population profiles.

One particular area where standard culture plates and plating methods may fail to properly detect the microbial population of a sample involves the culturing of L-form microorganisms (e.g., L-form bacteria). L-form bacteria, also sometimes referred to as pleomorphic, fastidious, intracellular, or cell-wall-deficient bacteria, are strains of bacteria that may normally exist with full cell wall structures in a planktonic environment, but which lack cell walls and/or reside intracellularly when in L-form. L-form bacteria can develop from Gram-positive as well as Gram-negative bacteria. L-form bacteria are often difficult to detect within clinical and/or environmental samples, and may be missed by standard laboratory procedures using standard culture plates.

According to some embodiments, a microbiological culture plate device, comprises: a base section having a bottom surface and a first sidewall coupled to the bottom surface and extending upwards and away from the bottom surface to define an interior space of the culture plate; a lid section configured in size and shape to selectively attach to the base section and enclose the interior space, the lid including an upper surface and a second sidewall extending downwards and away from the upper surface, the lid being configured in size and shape to fit upon the base section to enclose the interior space; an extension having an upper end and a lower end, the extension being mechanically coupled to the upper surface of the lid and extending downward from the upper surface to the lower end; and a cover attached to the lower end of the extension, the cover extending substantially horizontally from the extension, wherein the cover is configured to contact an upper surface of solid media placed within the interior space when the lid section is placed upon the base section.

In some embodiments, the base section includes solid media formed from agarose gel, collagen, laminin, elastin, peptidoglycan, fibronectin, or combinations thereof. In some embodiments, the base section is formed from one or more material selected from glass, ceramic, or polymer (e.g., polypropylene, polystyrene, polycarbonate, etc.), metal, or any combination thereof. In some embodiments, a base section is formed from a material that is capable of being sterilized (e.g., by autoclave, UV sterilization, chemical sterilization, etc.).

In some embodiments, the lid section has a diameter larger than a diameter of the base section.

In some embodiments, the cover is positioned so as to be closer to the upper surface of the lid section than a farthest extension of the second sidewall. In some embodiments, the cover is positioned so as to be substantially aligned with the center of the lid section. In some embodiments, the cover is formed from one or more material selected from glass, ceramic, or polymer (e.g., polypropylene, polystyrene, polycarbonate, etc.), metal, or any combination thereof. In some embodiments, a cover section is formed from a material that is capable of being sterilized (e.g., by autoclave, UV sterilization, chemical sterilization, etc.). In some embodiments, the cover is height-adjustable relative to the lid section.

In some embodiments, the extension has a diameter less than a diameter of the cover. In some embodiments, the diameter of the extension is substantially uniform. In some embodiments, the extension is deflectable so as to allow the cover to be moved closer to the upper surface of the lid section. In some embodiments, the extension is translatable relative to the upper surface of the lid section to enable adjusting of the height of the cover.

In some embodiments, a culture plate further comprises a vent. In some embodiments, the vent is disposed in the first and/or second sidewall. In some embodiments, the vent further comprises a semi-permeable membrane configured to allow passage of water out of the interior space of the culture plate while resisting entry of contaminants.

In some embodiments, a culture plate further comprises an anchor having a first side attached to the upper end of the extension and a second side attached to the upper surface of the lid section.

### BRIEF DESCRIPTION OF THE DRAWINGS

FIG. 1 shows a microbiological culture plate that provides hydrologic stability for microorganisms cultured using the plate;

FIG. 2 shows a cross-sectional view of the culture plate of FIG. 1 in an open position;

FIG. 3 shows a cross-sectional view of the culture plate of FIG. 1 in an open position;

FIG. 4 shows a cross-sectional view of another embodiment of a culture plate;

FIG. 5 shows an embodiment of a culture plate having a height-adjustable cover;

FIG. 6 shows an embodiment of a culture plate having a height-adjustable cover;

FIG. 7 shows an embodiment of a culture plate having a height-adjustable cover;

FIG. 8 shows an embodiment of a culture plate having a height-adjustable cover;

FIG. 9 shows an embodiment of a culture plate having a base section, a lid section, an extension, and a cover; and

FIG. 10 shows an embodiment of a culture plate that includes an anchor coupled to an extension opposite the cover.

### DETAILED DESCRIPTION

Aspects of the disclosure relate to microbiological culture plates and methods of using the same. The disclosure is based, in part, on microbiological culture plates configured for providing effective hydrostatic stabilization of inoculants added to the plates. Certain embodiments described herein are configured to beneficially maintain a hydrated state of the inoculant to therefore enable hydrologically sensitive microbes within the inoculant to be more effectively cultured. As compared to conventional microbiological culture plates and methods, embodiments described herein may provide more effective culturing of microorganisms that are often missed and/or are difficult to grow and detect, such as L-form microorganisms, for example L-form bacteria.

A sample having or suspected as potentially having L-form bacteria may be initially cultured in a liquid medium. However, plating of the L-form bacteria on solid media may be necessary to isolate strains within the sample. However, because L-form bacteria lack cell walls or have limited cell wall structures, the transition to solid media may prevent continued growth and culturing of the L-form bacteria. If L-form bacteria were present within the sample, they may go undetected when plated on conventional solid media plates.

In addition, it is often desirable to cause L-form bacteria present in an inoculant to revert to a “classic” (i.e., “cell-wall-sufficient”) morphology to enable characterization, storage, antibiotic testing, etc. At least some L-form bacteria will revert to a classic form when plated on solid media. However, under standard plating techniques using standard microbiological plate equipment, the transition from L-form to classic form may be prevented or limited. For example, without fully formed cell walls, the relatively fragile L-form bacteria may be unable to withstand the hydrologic changes occurring when moving from a liquid media environment to a solid media environment.

### Definitions

As used throughout this disclosure, the terms “cell-wall-sufficient bacteria” (“CWS bacteria”) or “classic-form bacteria” refer to strains of bacteria having a morphology with an identifiable and recognizable cell wall structure, such as the peptidoglycan layer of Gram positive bacteria and the relatively thinner peptidoglycan layer positioned between the cell membrane and the outer membrane (lipopolysaccharide layer) of Gram negative bacteria. As used herein, the term CWS bacteria also refers to mycobacteria, bacteria within the archaea domain, and other forms of bacteria known to those of skill in the art to typically exhibit a cell wall structure, even if not necessarily easily categorized as Gram positive or Gram negative bacteria.

The terms “L-form bacteria,” “pleomorphic bacteria,” “hidden bacteria,” “intracellular bacteria,” “fastidious bacteria,” and the like do not have standard definitions. The terms are often used synonymously in the art, but in some instances, for example, the term “intracellular bacteria” may refer to bacteria residing within a host cell regardless of level of cell wall formation of the bacteria.

As used herein, the term “L-form bacteria” refers to strains of bacteria often found to reside intracellularly within a host cell and which do not exhibit a full cell wall structure. Such bacteria are distinguished from typical cell-wall-sufficient bacteria for which traditional culturing and detection equipment and methods are directed. “L-form bacteria” include bacterial strains with morphologies lacking any identifiable cell wall structure or cell wall components, and include strains including an undeveloped or incomplete cell wall structure, such as strains containing some cell wall components but lacking sufficient structure to fully define the cell wall (e.g., strains with variable shape as opposed to typical cocci, rod, and/or spiral characterization). The skilled artisan will recognize that the term “L-form bacteria” does not, however, refer to bacteria of the genus Mycoplasma.

In some instances, the term “L-form bacteria” therefore includes strains of bacteria that do not yet include fully recognizable cell wall structures, but which are transitioning toward cell wall sufficient strains. The term “L-form bacteria” also refers to pleomorphic bacteria which are capable of progressing from a reduced-cell-wall or absent-cell-wall-form toward a classic form with a full cell wall. The term also includes species and/or strains of bacteria that are not known to exist in nature in a CWS form, but which have been found to reside in one or more samples in L-form.

The term “L-form capable bacteria” is used herein to describe bacteria that are found within an L-form sample and which have been cultured from the L-form morphology into a CWS morphology. Such strains often exhibit flexible morphological characteristics and are able to revert back to an L-form morphology under certain environmental conditions, such as when exposed to certain antibiotics and/or when immunological agents within an infected host.

Although the exemplary embodiments described herein refer specifically to bacteria, one of skill in the art will understand that certain principles disclosed herein may be utilized for culturing, screening, and/or detecting fungi (e.g., yeast), protozoans, and other pathogenic microorganisms capable of residing intracellularly within host cells and/or capable of being hidden from immune system responses within biological fluids or tissues.

As used herein, the term “sample” refers to a biological sample such as a tissue sample, whole blood sample, serum sample, plasma sample, and the like. Such samples are typically obtained from mammalian sources. As used herein, “sample” may also refer to mixtures containing the tissue/clinical sample. For example, a sample may be added to or mixed with a growth medium to promote the growth of bacteria within the sample. When such a mixture is further processed (e.g., transferred, analyzed, monitored, stored, etc.), the mixture may be referred to simply as the “sample.”

As used herein, the term “inoculant” refers to a sample having or suspected as potentially harboring bacteria. The inoculant may be derived from a biological sample such as a tissue sample, whole blood sample, serum sample, plasma sample, and the like. Such samples are typically obtained from mammalian sources and in particular are typically from human patients for use in a clinical setting. The “inoculant” may also refer to mixtures containing the tissue/clinical sample. For example, a sample may be added to or mixed with a growth medium to promote the growth of bacteria within the sample. When such a mixture is further processed (e.g., transferred to solid media plates, comminuted, stored, etc.), the mixture may be referred to simply as the “inoculant.”

### Culture Plates

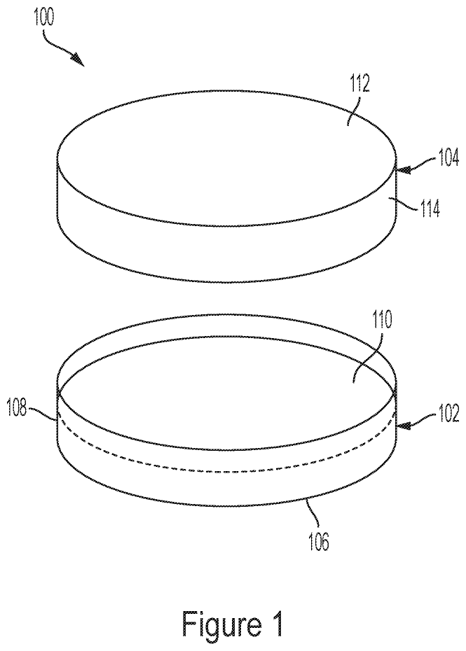

FIG. 1 illustrates an exemplary embodiment of a microbiological culture plate 100 configured to provide effective hydrologic stability for microorganisms cultured using the plate. The illustrated plate 100 includes a base section 102 and a lid section 104. The base section 102 includes a bottom surface 106 and a sidewall 108. The sidewall 108 is attached to the bottom surface 106 and extends upwards and away from the bottom surface 106 to form and define an interior space of the base section 102.

Solid media 110 may be held in the interior space of the base section 102. The solid media 110 may include agarose gel and/or another suitable gelling agent (e.g., collagen, laminin, elastin, peptidoglycan, fibronectin, combinations thereof). The solid media 110 may be a complex media, defined media, minimal media, selective media, or other microbiological culture media as known in the art.

The illustrated lid section 104 includes an upper surface 112 and a sidewall 114. The sidewall 114 is attached to the upper surface 112 and extends downward from the upper surface 112 to define an interior space of the lid section 104. The lid section 104 is configured in size and shape to be selectively positioned upon the base section 102 to enclose the solid media 110. Typically, the lid section 104 has a diameter slightly larger than the diameter of the base section 102 to allow the sidewall 114 of the lid section 104 to circumscribe the sidewall 108 of the base section when the lid section 104 is positioned upon the base section 102 to close the plate 100.

Alternative embodiments may include differently configured lids and/or base sections. For example, some embodiments may include lids having a smaller diameter than the base section such that the sidewall of the base section circumscribes the sidewall of the lid when the plate is closed. Some embodiments may include lids and base sections of substantially equal diameter such that the sidewall of the lid rests upon the sidewall of the base when the plate is closed.

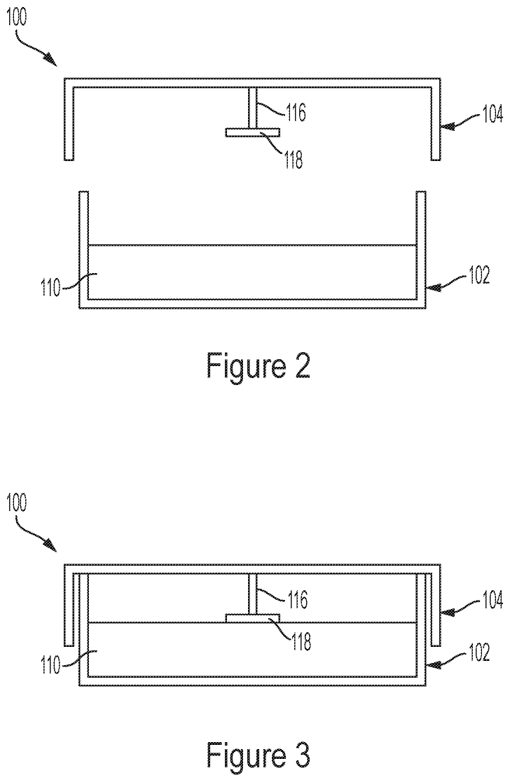

FIGS. 2 and 3 illustrate cross-sectional views of the culture plate 100 in an open position (i.e., with the lid section 104 detached from the base section 102) and in a closed position (i.e., with the lid section 104 placed upon the base section 102), respectively. As shown, the lid section 104 includes an extension 116 attached to the upper surface 112 and extending downward and away from the upper surface 112. A cover 118 is attached to the lower end of the extension 116. The cover 118 extends substantially horizontally outward from the point of attachment at the lower end of the extension 116.

As shown in FIG. 3, the cover 118 is positioned to contact or nearly contact the upper surface of the solid media 110 when the lid section 104 is placed upon the base section 102 to close the plate 100. In use, an inoculant may be placed on a portion of the solid media 110 corresponding to the cover 118, so that when the lid section 104 is placed upon the base section 102, the cover 118 is brought into contact with the inoculant. The cover 118 thereby functions to protect the inoculant and allows for effective transition of bacteria within the inoculant to growth on the solid media 110. In particular, the cover 118 functions to prevent overly rapid drying of the inoculant, allowing hydrologically sensitive bacteria such as L-form bacteria to transition to growth on the solid media 110.

In at least some circumstances, the cover 118 can also provide a zone where anaerobic and aerobic conditions balance to provide effective growth of classic-form bacterial colonies transitioning from L-form bacteria within the inoculant. Surprisingly, it has been found that the majority of such transitioned classic-form bacteria occurs at a “sweet zone” located underneath the cover 118 near the edge of the cover 118 (e.g., within between about 1 μm and about 1 cm from the edge of the cover). Without being bound to any particular theory, it is believed that the growth conditions in this region, including conditions related to moisture/hydration and oxygen exposure, are conducive to allowing L-form bacteria within the inoculant to transition to classic-form colonies on the solid media 110.

The cover 118 may be sized to provide a surface area that covers about 3% to about 50% (e.g., about 3%, 5%, 10%, 15%, 20%, 25%, 30%, 35%, 40%, 45%, or 50%) of the surface area of the upper surface of the solid media 110. In some embodiments, the cover 118 may be sized to provide a surface area that covers more than 50% (e.g., 55%, 60%, 70%, 80%, 90%, etc.) of the upper surface of the solid media 110. In preferred embodiments, the cover 118 is sized to provide a surface area of about 5% to about 20% (e.g., about 5%, 6%, 7%, 8%, 9%, 10%, 11%, 12%, 13%, 14%, 15%, 16%, 17%, 18%, 19%, or 20%) of the surface area of the upper surface of the solid media 110. It has been found that the use of covers within the foregoing ranges provides effective growth of bacteria from inoculant samples. A typical plate may have a diameter of about 3 to 4 inches (e.g., any diameter between 3 and 4 inches, inclusive). In some embodiments, a plate has a diameter between about 2 inches and 10 inches (e.g., about 1, 2, 3, 4, 5, 6, 7, 8, 9, or 10 inches in diameter). In some embodiments, a plate has a diameter between 10 mm and 250 cm (e.g., about 10 mm, 20 mm, 50 mm, 100 mm, 1 cm, 5 cm, 10 cm, 15 cm 20 cm, 25 cm, 50 cm, 75 cm, 100 cm, 150 cm, 200 cm, or 250 cm) in diameter. However, the principles and components described herein are not limited to plates within the standard size ranges, and may also be utilized in plates of smaller or larger size. The cover 118 may be formed, for example, from glass, ceramic, rigid polymer, film polymer, polypropylene, polystyrene, polycarbonate, metal, or combinations thereof.

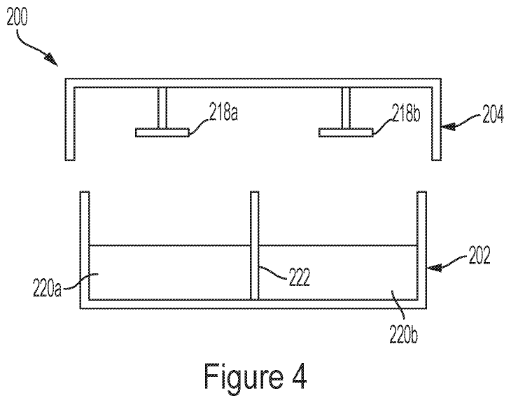

FIG. 4 illustrates a cross-sectional view of another embodiment of a culture plate 200 including a base section 202 and a lid section 204. In this embodiment, the lid section 204 includes two covers 218a, 218b and the base section 202 includes two corresponding growth zones 220a, 220b, optionally separated by a partition 222. The illustrated embodiment may be used with separately formulated solid media 210a, 210b and/or separate inoculants in each of the separate growth zones 220a, 220b. The components of the embodiment illustrated in FIG. 4 may otherwise be configured as the like components of the embodiment of FIGS. 1 through 3.

The number of covers and/or growth zones may be varied. The foregoing embodiments include one or two covers per plate. However, alternative embodiments may include more than two covers, including for example, tri-cover plates and quad-cover plates. Typically, where an embodiment includes two or more covers, the covers are arranged to be substantially evenly spaced on the lid structure to which they are attached.

Although the embodiments illustrated herein are shown with a circular shape, it will be understood that the same components and features may be utilized in culture plates having non-circular shapes, such as rectangular, square, other polygonal, oval, or irregular shapes.

In some embodiments, the cover is configured to have an adjustable height relative to the lid section. In this manner, the cover may be adjusted to provide desired contact with the surface of the solid media when the lid section is placed upon the base section. The depth of the base section can vary. In some embodiments, the depth of the base section ranges from about 1 mm deep to about 15 mm deep (e.g., about 1, 2, 3, 4, 5, 6, 7, 8, 9, 10, 11, 12, 13, 14 or 15 mm deep). When the base section of a plate is filled with media and the media is allowed to gel/solidify, the depth of the solid media may typically be about ⅛ of an inch (about 3-4 mm). However, the exact depth of the solid media may vary somewhat from plate to plate and/or from one use of a plate to another. If the depth of the solid media is too low (e.g., too shallow), for example, the cover may not reach the solid media when the lid section is placed on the base section. On the other hand, if the depth of the solid media is too high (e.g., too deep), the cover may be the first part of the lid section to contact the solid media, bearing all of the weight of the lid section and/or preventing the lid section from fully closing.

In some circumstances, it may not matter that the cover supports the weight of the lid section, so long as there is sufficient contact between the cover and the inoculant placed upon the solid media. However, in other circumstances it may be desirable to provide a height-adjustable cover. The height-adjustable cover embodiments disclosed below may be utilized with any of the other plate embodiments described herein, including those shown in FIGS. 1 through 4.

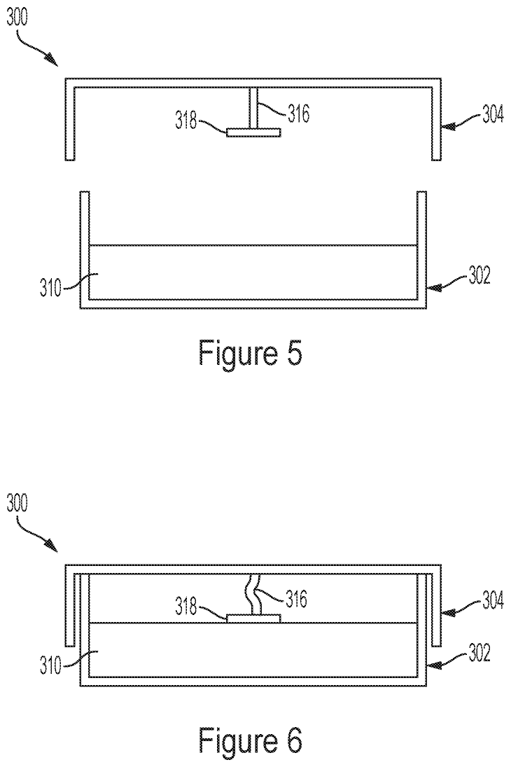

FIGS. 5 and 6 illustrate an embodiment of a culture plate 300 having a height-adjustable cover 318. FIG. 5 illustrates, in cross section, an example of a lid section 304 having an extension 316 coupled to a cover 318. In this embodiment, the extension 316 is formed of a material with sufficient flexibility to allow for deflection. The deflectable extension 316 may allow the cover 318 to be automatically positioned at the correct height when the plate is closed. As shown in FIG. 6, when the lid section 304 is placed upon a corresponding base section 302 and the cover 318 is brought into contact against the solid media 310, the deflectable extension 316 bends to allow the cover 318 to adjust position relative to the upper surface 312 of the lid section 304.

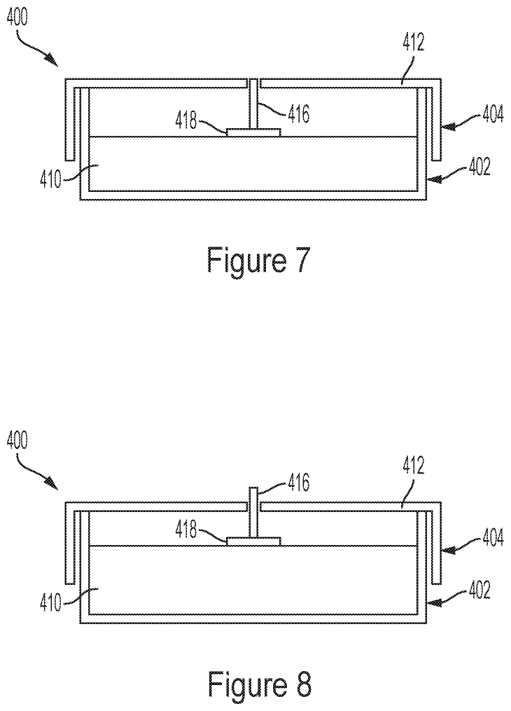

FIGS. 7 and 8 illustrate another embodiment of a culture plate 400 having a height-adjustable cover 418. In this embodiment, the extension 416 is connected to the upper surface 412 of the lid section 404 in a manner that allows the extension 416 to translate up and down relative to the upper surface 412. The distance between the upper surface 412 and the cover 418 can thereby be adjusted by raising or lowering the extension 416 relative to the upper surface 412. FIG. 7 illustrates a configuration where the solid media 410 is relatively shallow (e.g., low), and the extension 416 and cover 418 are placed in a low position to bring the cover 418 into contact with the solid media 410. FIG. 8 illustrates a configuration where the solid media 410 is relatively deep (e.g., high), and the extension 416 and cover 418 are placed in a relatively higher position to correspond to the greater height of the solid media 410.

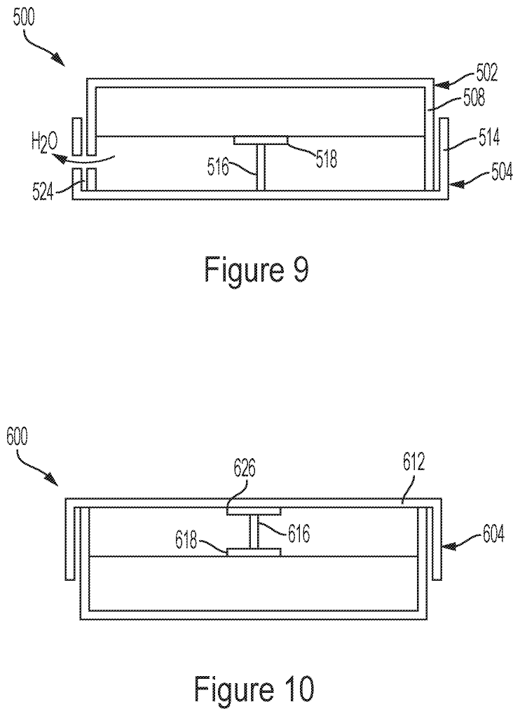

FIG. 9 illustrates another embodiment of a culture plate 500 having a base section 502, a lid section 504, an extension 516, and a cover 518. The plate 500 may include any of the features and components described above in relation to the embodiments of FIGS. 1 through 8. The illustrated embodiment also includes a vent 524 disposed on the sidewall 514 of the lid section 504, and which also may pass through a portion of the sidewall 508 of the base section 502, as shown. The vent 524 may additionally or alternatively be disposed on the upper surface 512 of the lid section 504. Although the illustrated embodiment includes a single vent 524, alternative embodiments may include multiple vents at multiple locations of the lid section 504.

The illustrated plate 500 is also shown in an “upside down” position, with the lid section 504 on the bottom and the base section 502 on top. In some circumstances, it may be beneficial to culture microorganisms with the plate in this position, at least for a portion of the overall culturing time period. This configuration places the surface of the solid media 510 in a downward facing position, and can aid in preventing the pooling of excessive moisture on the surface of the solid media 510. It has been found that by using a cover 518 positioned over an inoculant, and placing the plate 500 upside down (e.g., at least for the beginning portion of the culturing period), the plate avoids excessive pooling on the surface of the solid media 510 while also preventing the inoculant from drying too fast. In the particular application of using an inoculant having or suspected as potentially having L-form bacteria, this process has been shown to enable effective transition of the L-form bacteria within the inoculant to classic form colonies on the solid media 510.

The vent 524 may be configured to allow release of moist air and/or pooled water from the interior space of the culture plate 500. Removal of water can prevent buildup of excess water on the upper surface 512 of the lid section 502. By allowing for the release of moist air, the vent 524 can also provide moisture control in situations where the culture plate 500 is positioned right-side-up (instead of the illustrated upside-down position). In some embodiments, the vent 524 includes a semi-permeable membrane allowing water to escape but preventing contaminants from entering the interior space of the plate 500.

FIG. 10 illustrates another embodiment of a culture plate 600 that includes an anchor 626 coupled to the extension 616 opposite of the cover 618 to form a cover assembly 628. The anchor 626 may function to provide more effective attachment of the cover assembly 628 to the upper surface 612 of the lid section 604. As shown, the anchor 626 extends substantially horizontally from an end of the extension 616 opposite of the end to which the cover 618 is attached. The anchor 626 may enable more effective attachment to the upper surface 612 by providing sufficient surface area for attachment via one or more adhesives, plastic welding, and/or other suitable joining method. The culture plate 600 may also include any of the features and components described in relation to the other culture plate embodiments illustrated in FIGS. 1 through 9. Likewise, the cover assembly 628 illustrated in FIG. 10 may be utilized in any of the other embodiments described herein.

### Culture Methods

A culture plate as described in relation to any of FIGS. 1 through 10, or a culture plate using a combination of features from the separate embodiments shown in FIGS. 1 through 10, may be utilized to effectively perform microbial culture. In some embodiments, the sample or inoculant used to inoculate the culture plate has or is suspected as containing L-form bacteria. The culture plates and methods of using the culture plates described below are particularly beneficial for screening samples for the presence of L-form bacteria.

In some embodiments, a method of using a culture plate (any of the culture plates described in relation to FIGS. 1 through 10, or combinations thereof) includes a step of collecting a sample (e.g., a biological sample, such as a blood sample), a step of contacting the sample to a first, liquid growth medium, a step of incubating the inoculated first growth medium under a first set of incubation conditions, a step of transferring at least a portion of the inoculated first growth medium to a second, solid growth medium of the culture plate, and a step of incubating the culture plate second growth medium under a second set of incubation conditions to enable L-form bacteria within the sample/inoculant to grow to classic-form. In some embodiments, the steps involving the first, liquid growth medium are omitted and the sample is applied directly to the solid medium of the culture plate.

The step of collecting the sample may be performed using standard sample collection techniques, such as a blood draw, tissue swab, and the like. In some embodiments, the sample is collected in the same container in which the first growth medium is contained. Alternatively, the sample may be collected in one or more separate containers prior to storage, transport, and subsequent transfer to the container holding the first growth medium.

Various types and/or combinations of growth media may be used as the first and/or second growth media. For example, the media may be formulated as complex growth media (e.g., blood, yeast extract, bile, peptone, serum, and/or starch containing medias), defined growth media, or a selective media (e.g., nutrient selective for mannitol, cysteine, lactose, sucrose, salicin, xylose, lysine, or combinations thereof; selective based on carbon source, nitrogen source, energy source, and/or essential amino acids, lipids, vitamins, minerals, trace elements, or other nutrients; and/or selective antibiotic/antimicrobial containing media). Other exemplary growth media that may be used include R2A, nutrient, chocolate blood, blood, mannitol salt, Vogel Johnson, Kligler iron, Simmons citrate, Columbia, cetrimide, xylose-lysine-deoxycholate, tryptic soy, Tinsdale, Phenylethyl alcohol, Mueller-Hinton, MacConkey, brain-heart infusion (BHI), and lysogeny broth media. In one particular example, the first, liquid growth medium is serum (e.g., human, bovine) and/or brain-heart infusion (BHI) broth, and may be contacted with the sample as a liquid in suspension with the sample.

In preferred embodiments the growth media is formulated without substances that would hamper or restrict the growth of any bacteria found within the sample. For example, the growth media preferably omits antimicrobial enzymes (e.g., lysozyme, protease, etc.), antimicrobial peptides, and immune system components (e.g., leukocytes, complement system proteins, antibodies or other immunoglobulins, etc.).

For example, it has been discovered that L-form bacteria are often able to reside within a sample at a low-grade level without eliciting a full immune response and without converting to classic form. The presence of immune system components or other growth hampering substances within such samples can prevent the bacteria from being manifest in classic form, even though the bacteria are present within the sample in L-form. Under such circumstances, the removal or dilution of growth hampering substances and/or the transfer of L-form bacteria to growth media without growth hampering substances can promote progression of the bacteria within the sample to classic form, and thereby provide faster culture and screening of L-form bacteria within the sample.

In some embodiments, the first set of incubation conditions promote the aging of the sample tissue cells, allowing L-form bacteria present within the cells to grow. For example, as the cells die and rupture, more L-form bacteria are able to escape their intracellular positions and move into the surrounding extracellular medium. In addition, the dilution of the sample within the first growth medium dilutes the concentration of antibodies and other immune system components present within the blood sample, also enabling greater growth of the L-form bacteria.

In some embodiments, immune system components may be removed from the sample or from the inoculated first growth medium, or can be inactivated by adding an inactivating agent, such as a binding compound or complement inactivator, by adding one or more blocking antibodies, by washing, centrifuging, and/or filtering the sample to separate cells from other immune system molecules, or simply by diluting the sample sufficiently within the growth medium to render the components ineffective. In preferred embodiments, however, substances that would hamper polymerase chain reaction (PCR) or other analysis techniques (such as ethylenediaminetetraacetate (EDTA)), or that would inhibit conversion/reversion to classic form (such as EDTA), are omitted.

After the sample is contacted with the first growth medium, the method proceeds by incubating the inoculated first growth medium under a first set of incubation conditions. The collected sample is stored at a temperature about body temperature or at a temperature lower than about body temperature (e.g., about 98.6° F., about 37° C., etc.). For example, the collected sample may be stored at a temperature, constant or fluctuating, within a range or about 20° C. to about 40° C. (e.g., about 20° C., about 25° C., about 30° C., about 35° C., or about 40° C.), or within a range of about 25° C. to about 35° C. (e.g., about 25° C., about 26° C., 27° C., 28° C., 29° C., 30° C., 31° C., 32° C., 33° C., 34° C., or 35° C.), or more preferably within a range of about 25° C. to about 30° C., or about 27° C. In preferred embodiments, the inoculated first growth medium is stored at a temperature below body temperature.

It has been surprisingly found that L-form bacteria within a sample grow at a greater rate at temperatures lower than body temperature. For example, in human blood samples, which are typically stored at body temperature (37 degrees Celsius), it has been found that storage at a lower temperature increases the growth of L-form bacteria within the sample and enables L-form bacteria which would otherwise remain present at non-detectable levels to grow to observable levels. Preferably, incubation also omits rocking or shaking of the growth medium in order to reduce the amount of contact between any L-form bacteria and any antibodies or other immune components that may be present within the sample.

The inoculated first growth medium is incubated for a time sufficient to provide growth of any L-form bacteria present within the sample (e.g., for a time sufficient to allow any L-form bacteria present within the sample to achieve a detectable population). In some embodiments, this monitoring period can be about 120 hours or even longer than 120 hours. In more preferred embodiments, this monitoring period can be less than about 120 hours. For example, in some embodiments, the monitoring period can be within a range of about 24 hours to about 96 hours, or within a range of about 36 hours to about 84 hours. In other embodiments, the monitoring period is within a range of about 48 hours to about 72 hours.

The first growth medium may be monitored for the presence of any L-form bacteria by transferring the sample or a portion of the stored sample to a microscope slide, well plate, or other such apparatus allowing the microscopic visualization of the sample or portion of the sample. In preferred embodiments, in order to avoid the disruption of potentially fragile L-form bacteria within the sample or portion of the sample collected for microscopic inspection, the visual monitoring is carried out without traditional staining (e.g., Gram staining) or chemical or heat fixing steps. For example, the visual monitoring may be carried out by direct microscopic observation of the sample or portion thereof by preparing a wet-mount, live slide for observation. Although microscopy using live slides is the preferred manner of monitoring for L-form growth, other suitable monitoring techniques include spectrophotometric methods (including colorimetry and measurement of optical density), staining, and measurements of turbidity, total cellular DNA and/or protein levels, electrical field impedance, bioluminescence, carbon dioxide, oxygen, ATP production or consumption, and the like.

Preferably, the inoculated first growth medium is incubated until at least some (e.g., 10% or more, 25% or more, 50% or more, 75% or more, 90% or more) of the monitored cells of the sample have progressed to a state where they have ruptured to release intracellular L-form bacteria.

The second set of incubation conditions includes a temperature within a range of about 20° C. to about 40° C. (e.g., about 20° C., about 25° C., about 30° C., about 35° C., or about 40° C.). Preferably, the second growth medium is incubated at approximately body temperature (about 30° C. to 40° C. or about 37° C.). The second growth medium is incubated at this temperature for a time period of about 24 hours to 96 hours, or about 36 hours to 84 hours, or about 48 hours to 72 hours, or about 60 hours. In some embodiments, the temperature is then adjusted to a range that is below body temperature (e.g., about 25° C. to 35° C., or about 25° C. to 30° C., or about 27° C.) for a time period of about 4 days to about 30 days, or about 7 days to about 21 days, or about 14 days. In preferred embodiments, the temperature is adjusted to a range that is below body temperature for a time period of about 1-7 days, or about 3-5 days.

Although defined medias may be used as growth media in the methods described herein, it has been found that L-form bacteria are able to be efficiently cultured and detected using various complex medias such as BHI medias or those including serum (as the first and/or second growth medias). Beneficially, the methods described herein have enabled the screening of L-form bacteria without the need for generally more expensive defined medium formulations. Without being bound to any particular theory, it is thought that one or more process steps, such as the particular incubation conditions (e.g., time, temperature), transfer steps (e.g., transferring bacteria in a manner that enables bacteria within a sample to maintain a hydrated state), and/or culture plate construction enables L-form bacteria to be cultured without the need for custom-made or defined medias.

As growth occurs on the second growth medium in the culture plate, some strains of L-form bacteria may transition to classic form morphologies. Such bacteria may be transferred to separate media (e.g., one or more complex, selective, or defined medias described herein) until a single isolated strain is found on the media, and/or may be sampled and further analyzed according to well-known microbiological characterization techniques, including microscopic examination, staining (e.g., Gram, Malachite green/Safranin, and acid-fast stains), and selective growth testing. Other analytical techniques such as chromatography, gel separation, immunoassays, flow-through assays (e.g., plasmon resonance detection), fluorescent probe binding and measurement, automated cell/plate counting, microwell reading, DNA hybridization and amplification methods (e.g., polymerase chain reaction, strand displacement amplification), 16S rDNA sequencing, other molecular biological characterization techniques, and combinations thereof may also be used to analyze bacteria cultured or isolated using the methods described herein.

Beneficially, many of the bacteria cultured to a classic form using one of the culturing embodiments described herein maintain a flexible morphology capable of reverting back to L-form when exposed to appropriate conditions.

In some circumstances, it may be desirable to subject a sample to blending, vortexing, sonication, or other disruptive processes or combinations thereof in order to disassociate biofilms and/or aggregates, to rupture cells, or to otherwise disperse any bacteria and increase exposure to surrounding growth media prior to further screening. It has been surprisingly found that proper use of a comminution step in a screening process can increase yields, reduce culture times, and allow for faster detection and screening of samples having L-form bacteria. Although the exemplary method may be used to prepare any of the forms of samples defined above, it may be particularly useful in preparing samples known to contain, or known to be likely to contain, biofilms, root nodules, and/or other aggregates potentially harboring L-form growth.

After contacting the sample/inoculant to the solid medium of the culture plate, the sample/inoculant may be covered by the cover of the plate lid in order to maintain a hydrologically balanced state of the inoculant. It has been found that positioning the cover over the inoculant beneficially enables L-form bacteria within the inoculant to interface with the solid substrate to begin colonization of the solid medium. It is theorized that L-form bacteria are often in a hydraulically fragile state at this point in culturing (e.g., due to reduced or absent cell wall structures), and that excessive drying and/or too rapid concentrating of solutes within the inoculant can inhibit further culturing of the L-form bacteria.

In some embodiments, the inoculated culture plate is positioned for incubation with the inoculant side facing down (e.g., as shown in FIG. 9). In some embodiments, the method includes a step of incubating the solid medium for a first solid-phase incubation time period of about 4 hours to 24 hours, or about 6 hours to 18 hours, or about 12 hours.

As explained above, it has been discovered that greater culturing efficiency is made possible by maintaining a hydrated state of the inoculants and growth media as the disclosed methods are performed. For example, during the first solid-phase incubation time period, the relative humidity may be maintained within a range of about 40% to about 100% (e.g., about 40%, 50%, 60%, 70%, 80%, 90%, 95%, 99%, or 100%), or about 50% to about 90%, or about 60% to about 80%.

In some embodiments, the method further includes a step of repositioning the culture plate with the inoculant side up. It has been discovered that, at this point in the progression of L-form cultures, the L-form bacteria have typically progressed enough and/or the insert has sufficiently interfaced with the solid medium, such that the benefits of repositioning the solid medium to allow evaporation of water that has built up in the inverted position outweigh the detrimental effects, if any, of repositioning.

In some embodiments, the method further includes a step of incubating the solid medium for a second solid-phase incubation period. The second solid-phase incubation time period is preferably performed in an atmosphere having similar relative humidity levels of the first solid-phase incubation time period, and for a time period ranging from about 12 hours to about 84 hours, or about 24 hours to about 72 hours, or about 36 hours to about 60 hours, or about 48 hours. In some embodiments, one or more cultures are further incubated at a temperature in a range that is below body temperature (e.g., about 25° C. to about 35° C., or about 25° C. to about 30° C., or about 27° C.) for a time period of about 4 to about 30 days, or about 7 to 21 about days, or about 14 days. In preferred embodiments, the one or more cultures are further incubated at a temperature below body temperature for a period of about 1 to 7 about days, or about 3-5 days.

Additional details regarding methods of culturing L-form bacteria, transitioning L-form bacteria to classic form colonies, and screening samples for the presence of L-form bacteria are found in U.S. Patent Application Publication No. 2016/0168614, which is incorporated herein by this reference in its entirety.

What is claimed is:

1. A microbiological culture plate device, comprising:
a base section having a bottom surface and a first sidewall coupled to the bottom surface and extending upwards and away from the bottom surface to define an interior space of the culture plate;
a lid section configured in size and shape to selectively attach to the base section and enclose the interior space, the lid including an upper surface and a second sidewall extending downwards and away from the upper surface, the lid being configured in size and shape to fit upon the base section to enclose the interior space;
an extension having an upper end and a lower end, the extension being mechanically coupled to the upper surface of the lid and extending downward from the upper surface to the lower end; and
a cover attached to the lower end of the extension, the cover extending substantially horizontally from the extension, wherein the cover is configured to contact an upper surface of solid media placed within the interior space when the lid section is placed upon the base section.

2. The device of claim 1, wherein the base section includes solid media formed from agarose gel, collagen, laminin, elastin, peptidoglycan, fibronectin, or combinations thereof.

3. The device of claim 1 or 2, wherein the lid section has a diameter larger than a diameter of the base section.

4. The device of any one of claims 1 through 3, wherein the cover is positioned so as to be closer to the upper surface of the lid section than a farthest extension of the second sidewall.

5. The device of any one of claims 1 through 4, wherein the cover is positioned so as to be substantially aligned with the center of the lid section.

6. The device of any one of claims 1 through 5, wherein the cover is formed from one or more of glass, ceramic, or polymer material.

7. The device of any one of claims 1 through 6, wherein the extension has a diameter less than a diameter of the cover.

8. The device of any one of claims 1 through 7, wherein the diameter of the extension is substantially uniform.

9. The device of any one of claims 1 through 8, wherein the cover is height-adjustable relative to the lid section.

10. The device of claim 9, wherein the extension is deflectable so as to allow the cover to be moved closer to the upper surface of the lid section.

11. The device of claim 9 or claim 10, wherein the extension is translatable relative to the upper surface of the lid section to enable adjusting of the height of the cover.

12. The device of any one of claims 1 through 11, the culture plate further comprising a vent.

13. The device of claim 12, wherein the vent is disposed in the first and/or second sidewall.

14. The device of claim 12 or claim 13, the vent further comprising a semi-permeable membrane configured to allow passage of water out of the interior space of the culture plate while resisting entry of contaminants.

15. The device of any one of claims 1 through 14, further comprising an anchor having a first side attached to the upper end of the extension and a second side attached to the upper surface of the lid section.

16. A method of culturing bacteria while maintaining effective hydrologic stability, the method comprising:
providing a culture plate as in any one of claims 1 through 15;
applying an inoculant to the upper surface of the solid media;
positioning the lid section of the culture plate onto the base section of the culture plate to bring the cover into contact with the inoculant.

17. The method of claim 16, wherein the inoculant is a biological sample or is derived from a biological sample.

18. The method of claim 17, wherein the inoculant is mammalian blood or is derived from mammalian blood.

19. The method of any one of claims 16 through 18, further comprising, prior to applying the inoculant to the upper surface of the solid media, contacting a collected sample to a liquid growth medium and incubating the liquid growth medium, wherein the inoculant is derived from the liquid growth medium.

20. The method of any one of claims 16 through 19, further comprising incubating the culture plate for a first solid-phase incubation time period with the culture plate positioned inoculant-side-down, and subsequently incubating the culture plate for a second solid-phase incubation time period with the culture plate positioned inoculant-side-up.
